(19) United States
(12) Patent Application Publication
(10) Pub. No.: US 2016/0067070 A1
(43) Pub. Date: Mar. 10, 2016

(54) BIOERODIBLE POLYMERIC STENT SCAFFOLDING PATTERN

(71) Applicant: BOSTON SCIENTIFIC SCIMED, INC., Maple Grove, MN (US)

(72) Inventor: Dennis A. Boismier, Shorewood, MN (US)

(21) Appl. No.: 14/846,294

(22) Filed: Sep. 4, 2015

Related U.S. Application Data
(60) Provisional application No. 62/045,974, filed on Sep. 4, 2014.

(51) Int. Cl. A61F 2/915

(52) U.S. Cl. CPC A61F 2/915; A61F 2250/0098; A61F 2002/91525; A61F 2002/9155; A61F 2210/0004; A61F 2230/0069

(57) ABSTRACT

A stent includes a tubular network of struts cut from a bioerodible polymer tube. The tubular network includes a plurality of bands and a plurality of connectors. Each band includes at least nine peaks. Each band being connected to one or more adjacent bands by at least two connectors.

### CROSS-REFERENCE TO RELATED APPLICATIONS

This application claims priority under 35 U.S.C. §119 to U.S. Provisional Application Ser. No. 62/045,974, filed Sep. 4, 2014, the entirety of which is incorporated herein by reference.

### TECHNICAL FIELD

This invention relates to bioerodible polymeric stent, and more particularly to a scaffolding pattern for a bioerodible polymeric stent.

### BACKGROUND

Stents are generally cylindrically shaped devices, which function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of the diameter of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon_{—} angioplasty, stenting, or valvuloplasty) with apparent success.

The treatment of a diseased site or lesion with a stent involves both delivery and deployment of the stent. “Delivery” refers to introducing and transporting the stent through a bodily lumen to a region, such as a lesion, in a vessel that requires treatment. “Deployment” corresponds to the expanding of the stent within the lumen at the treatment region. Delivery and deployment of a stent are accomplished by positioning the stent about one end of a catheter, inserting the end of the catheter through the skin into a bodily lumen, advancing the catheter in the bodily lumen to a desired treatment location, expanding the stent at the treatment location, and removing the catheter from the lumen.

In the case of a balloon expandable stent, the stent is mounted about a balloon disposed on the catheter. Mounting the stent typically involves compressing or crimping the stent onto the balloon. The stent is then expanded by inflating the balloon. The balloon may then be deflated and the catheter withdrawn. In the case of a self expanding stent, the stent may be secured to the catheter via a retractable sheath or a sock. When the stent is in a desired bodily location, the sheath may be withdrawn which allows the stent to self expand.

The stent must be able to satisfy a number of mechanical requirements. First, the stent must be capable of withstanding the structural loads, namely radial compressive forces, imposed on the stent as it supports the walls of a vessel. Therefore, a stent must possess adequate radial strength. Radial strength, which is the ability of a stent to resist radial compressive forces, is due to strength and rigidity around a circumferential direction of the stent. Radial strength and rigidity, therefore, may also be described as, hoop or circumferential strength and rigidity.

Once expanded, the stent must adequately maintain its size and shape throughout its service life despite the various forces that may come to bear on it, including the cyclic loading induced by the beating heart. For example, a radially directed force may tend to cause a stent to recoil inward. Generally, it is desirable to minimize recoil.

In addition, the stent must possess sufficient ductility to allow for crimping, expansion, and cyclic loading. Longitudinal flexibility is important to allow the stent to be maneuvered through a tortuous vascular path and to enable it to conform to a deployment site that may not be linear or may be subject to flexure. Finally, the stent must be biocompatible so as not to trigger any adverse vascular responses.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). A conventional stent is allowed to expand and contract through movement of individual structural elements with respect to each other.

A medicated stent may be fabricated by coating the surface of either a metallic or polymeric scaffolding with a polymeric carrier that includes an active or bioactive agent or drug. Polymeric scaffolding may also serve as a carrier of an active agent or drug.

Frequently, only a temporary presence of the stent in the body is necessary to fulfill the medical purpose. Surgical intervention to remove stents, however, can cause complications and may not even be possible. One approach for avoiding a permanent presence of all or part of an endoprosthesis is to form all or part of the endoprosthesis out of bioerodible material. The term “bioerodible” as used herein is understood as the sum of microbial procedures or processes solely caused by the presence of endoprosthesis within a body, which results in a gradual erosion of the structure formed of the bioerodible material.

At a specific time, the stent, or at least the part of the stent that includes the bioerodible material, loses its mechanical integrity. The erosion products are mainly absorbed by the body, although small residues can remain under certain conditions. A variety of different bioerodible polymers (both natural and synthetic) and bioerodible metals (particularly magnesium and iron) have been developed and are under consideration as candidate materials for particular types of stents. Many of these bioerodible materials, however, have significant drawbacks. These drawbacks include the erosion products, both in type and in rate of release, as well as the mechanical properties of the material. Polymers have been used to make stent scaffolding, but a variety of factors that affect a polymeric stent's ability to retain its structural integrity when subjected to external loadings, such as crimping and balloon expansion forces. In comparison to metals, polymers typically have a low strength to weight ratio, which means that additional material is used to provide an equivalent mechanical property to that of a metal. Polymeric scaffolding can also be brittle or have limited fracture toughness. Anisotropic and rate-dependant inelastic properties (i.e., strength/stiffness of the material varies depending upon the rate at which the material is deformed) of polymeric materials can complicate the working of a polymeric material, particularly, a bioerodible polymer such as PLLA and PLGA.

### SUMMARY

A stent provided herein includes a tubular network of struts including a bierodible polymer. In some cases, the tubular network can cut from a bioerodible polymer tube. The tubular network can include a plurality of bands and a plurality of connectors, with each band including at least nine peaks, and with each band being connected to one or more adjacent bands by at least two connectors. In some cases, each band is connected to one or more adjacent bands by at least three connectors. In some cases, each band includes exactly nine peaks. In some cases, a stent having bands each having exactly nine peaks can have an outer diameter of between 2.0 mm and 5.0 mm when each peak is expanded to have a peak angle of 90 degrees for each peak. In some cases, each band includes more than nine peaks. For example, a band including ten peaks can have an outer diameter of 3.5 mm or greater when expanded to have a peak angle of 90 degrees for each peak.

Stents provided herein can include any suitable number of bands. In some cases, stents provided herein can include at least six bands including two end bands and at least four internal bands. In some cases, stents provided herein can include at least ten bands including two end bands and at least eight internal bands. In some cases, each end band is connected to an internal band by more than four or more connectors while each internal band is connected to at least one other internal band by three or fewer connectors. In some cases, each end band is connected to an internal band by nine connectors. In some cases, one or more connectors connecting an end band to an internal band includes a radiopaque marker. In some cases, stents provided herein include at least 3 radiopaque markers at each end of the stent. Stents provided herein can include connectors that connect two opposite peaks of adjacent bands.

Stents provided herein can having a wall thickness of less than 150 microns. In some cases, stents provided herein can have a wall thickness of less than 140 microns, less than 130 microns, less than 120 microns, less than 110 microns, or less than 100 microns. In some cases, a stent provided herein can have a wall thickness of about 120 microns.

The bands and connectors of a stent provided herein can be formed to have widths of between 180 and 250 microns. In some cases, bands and connectors of a stent provided herein can be formed to have a width of between 200 and 230 microns, between 180 and 200 microns, or between 230 and 250 microns. In some cases, peaks provided herein can be formed to have a wider width than other sections of the bands or the connectors. For example, peaks can be formed to have a width of between 230 and 250 microns and other portions of the bands and connectors can have a width of between 180 microns and 230 microns. In some cases, peaks can define an aperture there through. Stents provided herein can have any suitable peak width to strut width ratio. In some cases, each band is formed to have a peak width to strut width ratio of between 0.9 and 1.25. In some cases, each band is formed to have a peak width to strut width ratio of between 1.0 and 1.1 mm.

Stents provided herein can be crimped into a configuration adapted for delivery through a body lumen. In some cases, stents provided herein can have an expanded diameter of between 2.0 mm and 5.0 mm when each peak has a peak angle of 90 degrees and be crimped to a diameter of less than 1.4 mm. For example, a stent having an expanded diameter of about 3 mm when each peak is expanded to a peak angle of 90 degrees can be crimped to a crimped diameter of between 1.1 mm and 1.25 mm.

Stents provided herein can include any suitable bierodible polymer. In some cases, the bioerodible polymer can be selected from the group consisting of PLGA, PDLA, PLLA, PCL, PHBV, POE, PEO/PBTP, one or more polyamides, one or more polyanhides, and a combination thereof. In some cases, stents provided herein can include PLLA having a molecular weight of at least 30,000 Daltons. In some cases, stents provided herein can include PLLA having a Tg of at least 40° C. In some cases, stents provided herein can include PLLA having a molecular weight of at least 30,000 Daltons and a Tg of at least 40° C.

Stents provided herein provide suitable ductility to allow for crimping, expansion, and cyclic loading. Stents provided herein can provide improved longitudinal flexibility to allow the stent to be maneuvered through a tortuous vascular path and to enable it to conform to a deployment site that may not be linear or may be subject to flexure.

### DESCRIPTION OF DRAWINGS

FIG. 1 is a perspective view of a stent provided herein.

FIG. 2A depicts flat view of an outer diameter surface of a stent having a scaffolding pattern provided herein. FIG. 2B shows a detailed view of a section of the scaffolding pattern of FIG. 2A. FIG. 2C depicts a cross-sectional view of a strut of the scaffolding pattern of FIGS. 2A and 2B. FIG. 2D depicts a cross-sectional view of a stent having a scaffolding pattern depicted in FIGS. 2A-2C.

Like reference symbols in the various drawings indicate like elements.

### DETAILED DESCRIPTION

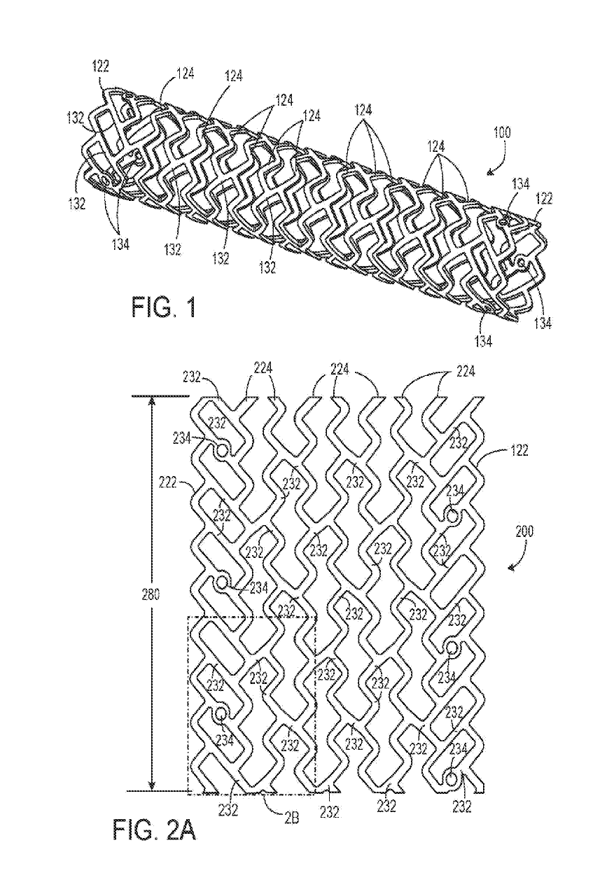

FIG. 1 depicts stent 100, an example of a stent provided herein. Stent 100 has a cylindrical shape. Stent 100 includes a plurality of bands, including end bands 122 and a plurality of internal bands 124. Each end band 122 and each internal band 124 includes nine peaks. Each internal band 124 is connected to two adjacent bands by a plurality of connectors 132. Each connector 132 extends peak-to-peak between adjacent bands. Each end band 122 is connected one adjacent internal band 124 by nine connectors 132. Internal bands 124 are each connected by three equally-spaced connectors 132. Select connectors 132 extending from each end band include radiopaque markers 134. Stent 100 can be a self-expandable stent or a balloon-expandable stent, or part of a stent-graft.

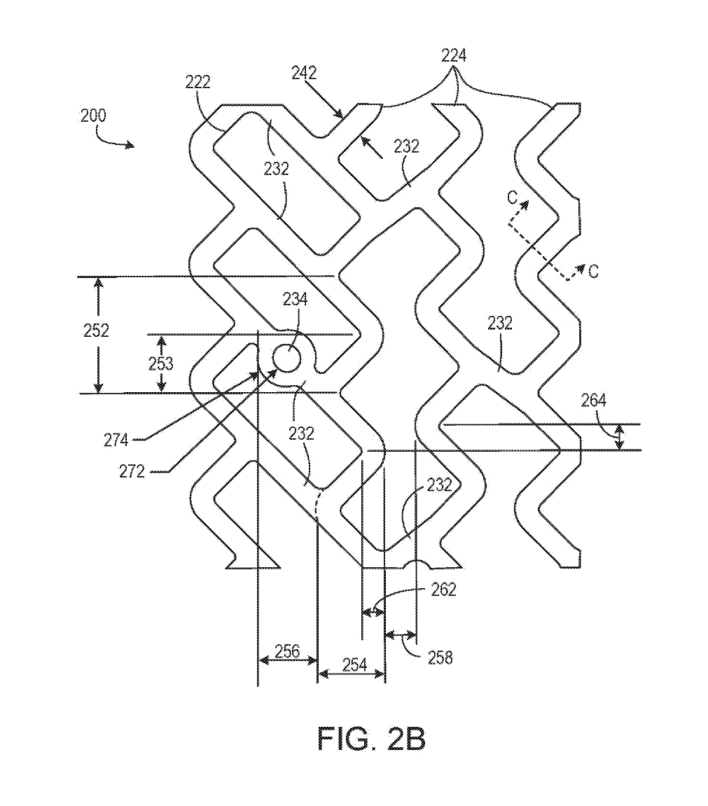

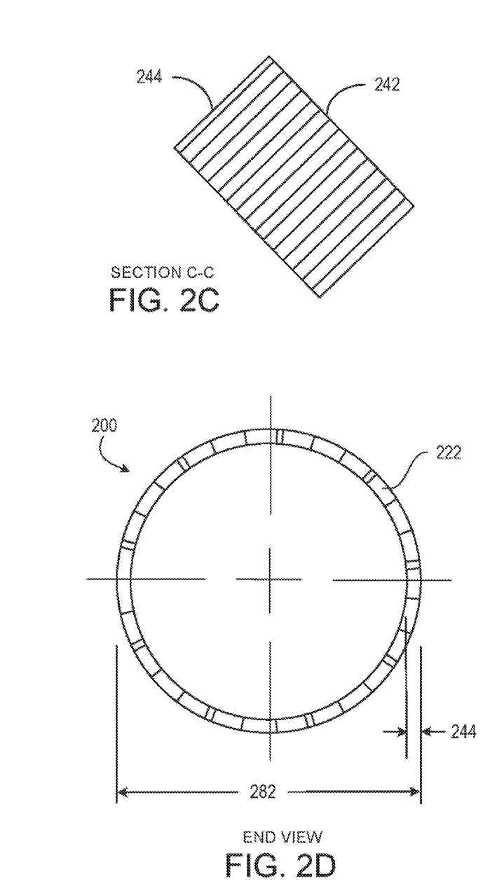

FIG. 2A depicts flat view of an outer diameter surface of a stent 200 having a scaffolding pattern provided herein. FIG. 2B shows a detailed view of section B of the scaffolding pattern of FIG. 2A. FIG. 2C depicts a cross-sectional view of a strut of the scaffolding pattern of FIGS. 2A and 2B along line C-C. FIG. 2D depicts a cross-sectional view of a stent having a scaffolding pattern depicted in FIGS. 2A-2C. As shown in FIG. 2D, stent 200 has a cylindrical shape. As shown in FIGS. 2A and 2B, stent 200 includes a plurality of bands, including end bands 222 and a plurality of internal bands 224. The stent scaffolding pattern of FIGS. 2A-2D differs from the stent depicted in FIG. 1 by the number of internal bands. FIG. 2A depicts seven internal bands 224. FIG. 1 depicts 16 internal bands 124. Stents provided herein can include any number of internal bands, which can be selected based on the desired length of the stent. In some cases, stents provided herein include at least 4 internal bands, at least 6 internal bands, at least 8 internal bands, at least 10 internal bands, at least 15 internal bands, at least 20 internal bands, or at least 25 internal bands.

As shown in FIG. 2A, similar to that shown in FIG. 1, each end band 222 and each internal band 224 includes nine peaks. Stents provided herein can include at least 9 peaks. In some cases, stents provided herein can include ten peaks, eleven peaks, or twelve peaks. For example, a stent having an expanded diameter of 4.0 mm or larger when each peak is expanded to a peak angle of 90 degrees can be designed to have ten peaks. FIG. 2A depicts a stent having an outer circumference 280 of 0.37102209 inches (about 9.434 mm), which is equal to the outer diameter 282 times pi. As shown in FIG. 2D, the outer diameter 282 is 0.1181 inches (about 3.0 mm). Although FIGS. 2A-2D depict a stent having an outer diameter of about 3.0 mm when each peak is expanded to a peak angle of 90 degrees, stents provided herein can have any suitable expanded diameter. As used herein, expanded diameter refers to a diameter of the stent when each peak is expanded to a peak angle of 90 degrees. In some cases, a nominal diameter used to describe a stent provided herein can be approximately equal to or less than the expanded diameter. In some cases, stents provided herein can have an expanded diameter of between 2.0 mm and 5.0 mm. In some cases, stents provided herein can have expanded diameters of between 2.5 mm and 4.0 mm. In some cases, stents provided herein can having expanded diameters of about 2.5 mm, about 2.75 mm, about 3.0 mm, about 3.5 mm, or about 4.0 mm. Stents provided herein can be crimped down to a crimped diameter. In some cases, the ratio of the expanded diameter to the crimped diameter can be at least 2.0, at least 2.25, at least 2.5, at least 3.0, or at least 3.5.

As shown in FIGS. 2A and 2B, each internal band 224 is connected to two adjacent bands by two or more connectors 232. As shown, each connector 232 extends peak-to-peak between adjacent bands. As shown in FIGS. 2A and 2B, the connection between each internal band 224 to another internal band 224 includes three equally-spaced connectors 232. In some cases, stents provided herein can include three or more connectors between adjacent internal bands. In some cases, stents provided herein have between three and five connectors between adjacent internal bands. In some cases, stents provided herein have between three and four connectors between adjacent internal bands. In some cases, stents provided herein have exactly three connectors between adjacent internal bands.

As shown in FIG. 2A, each end band 222 is connected to one adjacent internal band 224 by nine connectors 232. As shown, each connector 232 extends peak-to-peak between adjacent bands. In some cases, stents provided herein can include at least three connectors connecting each end band to an adjacent internal band. In some cases, stents provided herein can include at least four connectors connecting each end band to an adjacent internal band. In some cases, stents provided herein can include at least six connectors connecting each end band to an adjacent internal band. In some cases, stents provided herein can include at least 8 connectors connecting each end band to an adjacent internal band. In some cases, stents provided herein can include at least nine connectors connecting each end band to an adjacent internal band. In some cases, stents provided herein can include additional connectors connecting each end to an adjacent internal band as compared to the number of connectors connecting adjacent internal bands. The inclusion of additional connectors for each end band can increase the stiffness of the ends of the stent, but allow more flexibility in middle sections of the stent.

As shown in FIGS. 2A and 2B, select connectors 232 extending from each end band include radiopaque markers 234. As shown in FIG. 2A, each end of stent 200 can include three equally spaced radiopaque markers 234. Select connectors 234 can be formed (e.g., cut from a tube) to include one or more holding features adapted to retain a radiopaque marker 234. As shown, a holding feature can include a ring in the connector 232 with an aperture sized to secure a radiopaque marker 234. As shown in FIG. 2B, a ring for holding radiopaque marker 234 can have an inner diameter of between 0.2 and 0.3 mm and an outer diameter of between 4.5 and 6.0 mm, to hold a radiopaque marker 234 having an outer diameter equal to or greater than the inner diameter of the ring in order to achieve a snug fit. Radiopaque marker 234 can have any suitable shape. In some cases, radiopaque markers can be cylindrical. In some cases, radiopaque markers have a thickness approximately equal to the thickness of the stent wall. In some cases, radiopaque markers have a thickness greater than the thickness of the stent wall. Radiopaque markers provided herein can use any suitable material having a high visibility on imaging equipment. In some cases, the radiopaque marker can be biostable. In some cases, the radiopaque marker can be bioerodible. In some cases, radiopaque markers can include platinum, palladium, rhodium, iridium, osmium, ruthenium, tungsten, tantalum, rhenium, silver, and/or gold.

Stents provided herein can have any suitable stent wall thickness. As shown in FIGS. 2C and 2D, stent 200 has a wall thickness 244 of 0.0050 inches (about 127 microns). In some cases, stents provided herein can have wall thicknesses 244 of less than 150 microns, less than 140 microns, less than 130 microns, less than 125 microns, less than 120 microns, less than 100 microns, or less than 80 microns. In some cases, stents provided herein can have wall thicknesses 244 of at least 50 microns, at least 75 microns, at least 100 microns, at least 120 microns, or at least 125 microns. Stent wall thicknesses provided herein can reduce the risk of thrombus formation and improve healing times.

Stents provided herein can have struts having any suitable width. Referring to FIGS. 2B and 2C, struts of stent 200 can have a width 242 that is greater than the wall thickness 244. As shown in FIG. 2B, stent 200 can have struts having a width of 0.0080 inches (about 0.20 mm). In some cases, strut widths can be between 0.1 mm and 0.3 mm, between 0.15 mm and 0.25 mm, or between 0.18 mm and 0.22 mm. Strut widths provided herein can provide radial strength for the bioerodible polymeric stents provided herein. A ratio of the strut width to the wall thickness can be between 1.0 and 2.0, between 1.2 and 1.9, between 1.4 and 1.8, between 1.5 and 1.7, or be about 1.6. Strut width to wall thicknesses provided herein can provide enhanced radial strength. Stents provided herein can also have an offset between peaks in adjacent bands.

Stents provided herein can have any suitable offset. In some cases, stents provided herein can have a peak offset of between 0.1 mm and 0.4 mm, between 0.15 mm and 0.3 mm, or between 0.2 mm and 0.25 mm. FIG. 2B depicts an exemplary peak offset 264 between adjacent bands. An offset provided herein can provide clearance from interference during implantation and/or bending, as adjacent bands can abut. The distance between peaks in adjacent bands can also be any suitable value. As shown in FIG. 2B, a peak spacing 258 can be larger than the peak offset. In some cases, a peak spacing can be less than or equal to the peak offset. In some cases, stents provided herein can have a peak spacing of between 0.1 mm and 0.5 mm, between 0.2 mm and 0.4 mm, or between 0.25 mm and 0.35 mm. In other cases, stents provided herein have an offset of less than 0.1 mm. In some cases, stents provided herein can have no peak offset.

Stents provided herein can have any suitable ratio of peak width to strut width. As shown in FIG. 2B, peak width 262 can be about 0.0080 inches (about 0.2 mm), which yields a peak width to strut width ratio of about 1:1. In some cases, a ratio of peak width to strut width can be between 1:1.5 to 1.5:1, between 1:1.2 to 1.2:1, or between 1:1.1 and 1.1:1. FIG. 2B further depicts other dimensions of the stent design, such as dimensions 252, 253, 256, and 254, which are listed in inches. As shown, FIGS. 2A-2D show stent 200 in an expanded state after forming the bands and connector (e.g., by cutting a tube), at the expanded diameter 282, which shows the struts forming approximate 90 degree angles at the peaks. Stents provided herein, however, can be crimped to a smaller diameter such that angles of less than 45 degrees, less than 30 degrees, less than 20 degrees, less than 10 degrees, or less than 5 degrees are formed at each peak. Moreover, when in use, stents provided herein can be expanded past the expanded diameter. In general, stents 100 and 200 are designed to be radially compressed to allow for percutaneous delivery through an anatomical lumen, then deployed for implantation at the desired segment of the anatomical lumen. As used herein, deployment of the stent refers to radial expansion of the stent to implant the stent in the patient. The stresses involved during compression and deployment are generally distributed throughout various structural elements of the stent pattern.

The pattern of stents provided herein can allow for radial expansion and compression and longitudinal flexure. The pattern includes struts that are straight or relatively straight and bending elements. Bending elements bend inward when a stent is crimped to allow radial compression of the stent in preparation for delivery through an anatomical lumen. Bending elements also bend outward when a stent is deployed to allow for radial expansion of the stent within the anatomical lumen. After deployment, stents provided herein can be subjected to static and cyclic compressive loads from the vessel walls. Thus, bending elements may deform during use.

### Bioerodible Polymer

Stents provided herein include a bioerodible polymer. In some cases, stents provided herein are bioerodible. In some cases, bioerodible polymer in a stent provided herein is the primary source of the radial strength of the stent. In some cases, stents provided herein are completely or primarily composed of bioerodible polymer. In some cases, bands of stents provided herein are substantially free metallic material. In some cases, only radiopaque markers include metallic materials.

Stents provided herein can include any suitable bierodible polymer. In some cases, the bioerodible polymer can be selected from the group consisting of poly(lactide-co-glycolide) (PLGA), poly(D,L-lactic acid) (PDLA), poly(L-lactic acid) (PLLA), poly(caprolactone) (PCL), polyhydroxy-butyrate/-valerate copolymer (PHBV), polyorthoester (POE), polyethyleneoxide/polybutylene terephthalate copolymer (PEO/PBTP), one or more polyamides (such as Nylon 66 and polycaprolactam), one or more polyanhidride, and a combination thereof. In some cases, stents provided herein can include PLLA having a molecular weight of at least 30,000 Daltons. In some cases, stents provided herein can include PLLA having a Tg of at least 40° C. In some cases, stents provided herein can include PLLA having a molecular weight of at least 30,000 Daltons and a Tg of at least 40° C. Additional examples of polymers that may be used to fabricate a stent provided herein include, but are not limited to, poly(N-acetylglucosamine) (Chitin), Chitosan, poly(hydroxyvalerate), poly(hydroxybutyrate), poly(hydroxybutyrate-co-valerate), polyanhydride, poly(glycolic acid), poly(glycolide), poly(L-lactide), poly(D,L-lactic acid), poly(D,L-lactide), polyester amide, poly(glycolic acid-co-trimethylene carbonate), co-poly (ether-esters) (e.g. PEO/PLA), polyphosphazenes, and biomolecules (such as fibrin, fibrinogen, cellulose, starch, collagen and hyaluronic acid). Another type of polymer based on poly(lactic acid) that can be used includes graft copolymers, and block copolymers, such as AB block-copolymers (“diblock-copolymers”) or ABA block-copolymers (“triblock-copolymers”), or mixtures thereof.

Bioerodible polymers used in stents provided herein can be completely amorphous, partially crystalline, or almost completely crystalline. A partially crystalline polymer includes crystalline regions separated by amorphous regions. The crystalline regions do not necessarily have the same or similar orientation of polymer chains. However, a high degree of orientation of crystallites may be induced by applying stress to a semi-crystalline polymer. The stress may also induce orientation in the amorphous regions. An oriented amorphous region also tends to have high strength and high modulus along an axis of alignment of polymer chains. Additionally, for some polymers under some conditions, induced alignment in an amorphous polymer may be accompanied by crystallization of the amorphous polymer into an ordered structure. This is known as stress induced crystallization.

### Fabrication and Use

Stents provided herein, such as stents 100 and 200, may be fabricated from a polymeric tube or a polymeric sheet that has been rolled and bonded to form a tube. For example, the stent pattern may be formed on the polymeric tube or sheet by laser cutting away portions of the tube or sheet, leaving only struts and other members that function as scaffolding to support the walls of an anatomical lumen. Representative examples of lasers that may be used include, but are not limited to excimer, carbon dioxide, and YAG. In some cases, chemical etching may be used to form a pattern on a tube.

In some embodiments, a stent substrate in the form of a polymeric tube may be deformed by blow molding. In blow molding, the tube can be radially deformed or expanded by increasing a pressure in the tube by conveying a fluid into the tube. The fluid may be a gas, such as air, nitrogen, oxygen, or argon. The polymer tube may be deformed or extended axially by applying a tensile force by a tension source at one end while holding the other end stationary. Alternatively, a tensile force may be applied at both ends of the tube. The tube may be axially extended before, during, and/or after radial expansion.

Polymer chains in a stent substrate may initially have a preferential orientation in the axial direction as a result of extrusion, injection molding, tensile loading, machining, or other process used to form the stent substrate. In some cases, radial expansion of a stent substrate having polymer chains with an initial axial orientation will reorient or induce the polymer chains to have a circumferential orientation. In a biaxial orientation, the polymer chains are oriented in a direction that is neither preferentially circumferential nor preferentially axial. In this way, polymer chains can be oriented in a direction substantially parallel to the lengthwise axis of individual stent struts so as to increase the overall radial strength of the stent.

Optionally, after making the stent pattern, the stent may be crimped onto a balloon catheter or other stent delivery device. Prior to or during crimping, the stent may be heated to a crimping temperature Tc. In some embodiments, Tc is greater than ambient room temperature Ta to minimize or prevent outward recoil of the stent to a larger diameter after crimping. Outward recoil undesirably increases the delivery profile of the stent and may cause the stent to prematurely detach from the catheter during delivery to a target treatment site within a vessel. Also, Tc can be below Tg to reduce or eliminate stress relaxation during crimping. Stress relaxation during or after crimping leads to a greater probability of cracking during subsequent deployment of the stent. To reduce or prevent such cracking, the difference between Tc and Tg can be maximized by increasing Tg through stress induced crystallization.

After manufacturing, the stent can be deployed inside a blood vessel from a crimped diameter to a deployed outer diameter. In some cases, the deployed outer diameter is less than the expanded diameter. If the stent was crimped onto a balloon catheter, the deployment of the stent can include inflating the balloon catheter to urge the stent to move from its crimped configuration to an expanded, deployed configuration. In some cases, the stent may be self-expanding and deployment of the stent can include removing a sheath or other constraining device from around the stent to allow the stent to self-expand.

A number of embodiments of the invention have been described. Nevertheless, it will be understood that various modifications may be made without departing from the spirit and scope of the invention. Accordingly, other embodiments are within the scope of the following claims.

What is claimed is:

1. A stent comprising a tubular network of struts comprising a bioerodible polymer, the tubular network comprising a plurality of bands and a plurality of connectors, each band comprising at least nine peaks, each band being connected to one or more adjacent bands by at least two connectors.

2. The stent of claim 1, wherein each band is connected to one or more adjacent bands by at least three connectors.

3. The stent of claim 1, wherein each band consists of nine peaks.

4. The stent of claim 1, wherein the tubular network has a wall thickness of less than 150 microns.

5. The stent of claim 1, wherein the tubular network has a wall thickness of about 120 microns.

6. The stent of claim 1, wherein the bands and connectors each having a width of between 180 and 250 microns.

7. The stent of claim 1, wherein the stent comprises at least six bands including two end bands and at least four internal bands.

8. The stent of claim 7, wherein each end band is connected to an internal band by more than four or more connectors, wherein each internal band is connected to at least one other internal band by only 3 connectors.

9. The stent of claim 8, wherein each end band is connected to an internal band by nine connectors.

10. The stent of claim 7, wherein one or more connectors connecting an end band to an internal band includes a radiopaque marker.

11. The stent of claim 1, wherein each connector connects two opposite peaks of adjacent bands.

12. The stent of claim 1, wherein each band has a peak width to strut width ratio of between 0.9 and 1.25.

13. The stent of claim 1, wherein the stent is crimped to a crimped diameter of less than 1.40 mm and an expanded diameter of between 2.0 mm and 5.0 mm when each peak is expanded to an angle of 90 degrees

14. The stent of claim 13, wherein the crimped diameter is between 1.1 mm and 1.25 mm and the expanded diameter is about 3 mm when each peak is expanded to an angle of 90 degrees.

15. The stent of claim 13, wherein each band includes at least 10 peaks and the expanded diameter is at least 3.5 mm when each peak is expanded to an angle of 90 degrees.

16. The stent of claim 1, wherein the bioerodible polymer comprise a polymer selected from the group consisting of PLGA, PDLA, PLLA, PCL, PHBV, POE, PEO/PBTP, one or more polyamides, one or more polyanhides, or a combination thereof.

17. The stent of claim 1, wherein the bioerodible polymer comprises PLLA having a molecular weight of at least 30,000 Daltons.

18. The stent of claim 1, wherein the bioerodible polymer comprises PLLA having and a Tg of at least 40° C.

19. The stent of claim 1, wherein the stent includes at least 3 radiopaque markers at each end of the stent.

20. A stent comprising:
a tubular network of struts cut from a tube comprising PLLA, the tube having a wall thickness of 120 microns or less, the tubular network comprising at least six bands each having nine peaks and at least three connectors extending between and connecting opposite peaks of adjacent bands, each band or connector having a width of between 180 microns and 230 microns, the stent having a nominal diameter of 3.0 mm and being crimped to a crimped diameter of between 1.1 mm and 1.25 mm.
